(19) United States
(12) Patent Application Publication
(10) Pub. No.: US 2025/0152342 A1
(43) Pub. Date: May 15, 2025

(54) IMPLANTABLE PROSTHETIC VALVES WITH WOVEN VALVE CUSPS

(71) Applicant: RHEINISCH-WESTFÄLISCHE TECHNISCHE HOCHSCHULE (RWTH) AACHEN, Aachen (DE)

(72) Inventor: Thomas SCHMITZ-RODE, Aachen (DE)

(73) Assignee: RHEINISCH-WESTFÄLISCHE TECHNISCHE HOCHSCHULE (RWTH) AACHEN, Aachen (DE)

(21) Appl. No.: 18/723,047

(22) PCT Filed: Dec. 14, 2022
(86) PCT No.: PCT/EP2022/085946

§ 371 Date: Jun. 21, 2024

(30) Foreign Application Priority Data
Dec. 23, 2021 (DE) 10 2021 134 525.1

(51) Int. Cl. A61F 2/24

(52) U.S. Cl. CPC A61F 2/2418; A61F 2220/0025; A61F 2250/0017; A61F 2250/0018; A61F 2250/0028

(57) ABSTRACT

The present invention relates to a valve prosthesis for implantation in the human or animal body comprising at least one valve stent with an annular, lower valve base, at least two commissural struts extending from the valve base and valve cusps arranged on the valve stent, wherein the valve cusps are formed from one or more woven ribbon fabrics with approximately tangential warp threads and approximately axial weft threads with respect to the axis of symmetry of the annular flap base, wherein at least the ribbon fabric weft threads at the end of the ribbon fabric opposite the valve base reverse their orientation and form a free cusp edge.

The present invention relates to a valve prosthesis for implantation in the human or animal body comprising at least one valve stent with an annular, lower valve base, at least two commissural struts extending from the valve base and valve cusps arranged on the valve stent, wherein the valve cusps are formed from one or more woven ribbon fabrics with approximately tangential warp threads and approximately axial weft threads with respect to the axis of symmetry of the annular valve base, wherein at least the ribbon fabric weft threads at the end of the ribbon fabric opposite the valve base reverse their orientation and form a free cusp edge.

In recent decades in particular, medical progress has made it possible to significantly extend the time available with an almost unchanged quality of life. Artificial replacement materials and parts that can be made from them, which are able to temporarily or permanently take over the functions of parts of the body that do not work at all or only insufficiently, have made a significant contribution to improving quality of life. Examples include prostheses, which can be designed either as static supports, for example in the hip area, or as movable functional carriers, for example in the form of knee prostheses or heart valves. The latter prostheses in particular are subject to high technical requirements, as moving parts are exposed to a changing biomechanical load spectrum. The above applies even more to heart valve prostheses, which must provide sufficient fatigue strength under mechanical stress for an estimated 150 million load cycles over a period of 5 years. Animal substitute materials in the form of porcine heart valves or porcine and bovine pericardial patches are usually used as valve cusps to take over the functions. After decellularization, these materials essentially consist of collagen, which was designed by nature to provide the required long-term stability in the form of strength and elasticity. However, the disadvantage of these biocompatible materials is that they tend to accumulate and store calcium under in-vivo conditions in the human body, which inevitably contributes to the stiffening and embrittlement of the valves over time. This disadvantage generally leads to a reduced service life of the prostheses used. For this reason, alternative materials with suitable mechanical and biological properties that can be produced in a standardized and reproducible manner would be desirable. These materials should therefore not consist of animal materials, should not incorporate or accumulate functionally impairing endogenous substances and, ideally, should exhibit minor variations in terms of mechanical properties.

Different technical solutions for valve prostheses are also described in the patent literature.

For example, EP 3 139 861 B1 discloses a method of manufacturing a prosthetic flap capable of assuming a first shape in which the flap is open and a second shape in which the flap is closed, the flap comprising a sail assembly having at least one sail attached to a support member, the sail having a free edge capable of moving between a first position in which the flap assumes the first shape and a second position in which the flap assumes the second shape, the method comprising the following: providing a textile structure; forming the sail assembly from the textile structure such that an edge of the textile structure forms the free edge of the sail; wherein forming the sail assembly comprises joining two side edges of a single piece of the textile material to form a substantially tubular structure, wherein the inner layer forms the sail and the outer layers form the support member.

In another patent document, US 2011 153 008 A1, a valve endoprosthesis is disclosed which essentially comprises an expandable stent or frame made of several parts, i.e. an upper cylinder, a lower portion in the shape of a truncated cone and having a maximum diameter higher than that of the aortic annulus and decreasing to the diameter of the expandable stent or frame towards the proximal end, and arches, the upper cylinder being connected to the lower bearing portion via brackets and connected to said stent by stitches, staples or clips to a valve. The invention can be used in particular in the field of medicine, especially in plastic surgery and in particular in cardiac surgery, especially for cardiac prostheses.

Furthermore, WO 2010 020 660 A1 discloses an implantable valve prosthesis comprising at least one valve leaf and produced from a material structure of unidirectional reinforcing elements made of stretched ultra-high molecular weight polyolefin, extending in at least two directions, the reinforcing elements consisting of polyolefin having a modulus of elasticity of at least 60 GPa, characterized in that the material structure consists of exactly one weave or knitted mesh of the reinforcing elements made of stretched ultra-high molecular weight polyolefin.

Such solutions known from the prior art may offer further potential for improvement, especially with regard to long-lasting and consistent mechanical properties under in-vivo conditions.

It is the task of the present invention to at least partially overcome the disadvantages known from the prior art. In particular, it is the task of the present invention to provide a solution by which an improved mechanical load-bearing capacity of the valve prosthesis is achieved under in-vivo conditions and thus the fatigue strength of the prosthesis is extended.

According to the invention, the problem is solved by a flap prosthesis with the features of claim 1. Preferred embodiments of the invention are described in the dependent claims, in the description or in the figures, whereby further features described or shown in the dependent claims or in the description or in the figures may constitute an object of the invention individually or in any combination, unless the opposite is clearly apparent from the context.

According to the invention, a valve prosthesis for implantation in the human or animal body is thus at least comprising a valve stent with an annular, lower valve base, at least two commissural struts extending from the valve base and valve cusps arranged on the valve stent, wherein the valve cusps are formed from one or more woven ribbon fabrics with, in relation to the axis of symmetry of the annular flap base, approximately tangential warp threads and approximately axial weft threads, wherein at least the ribbon fabric weft threads at the end of the ribbon fabric opposite the flap base reverse their orientation and form a free cusp edge.

Surprisingly, it was found that the above-mentioned structure of valve stent and ligament fabric enables an improved biomechanical load transfer of the tensile load from the outside into the commissure struts when the valve is closed. Without being bound by theory, this is attributed to the fact that the warp and weft course bearing the main load, with the special alignment in relation to the valve stent and in particular to the commissural struts arranged on it, leads to improved long-term mechanical behavior under in vivo conditions. In addition to the alignment of the warp and weft threads, the “smooth” finish of the free cusp edge can also contribute synergistically to the improved closing behavior of the valve. The biomechanical forces that occur can be transferred more reliably into the valve stent, resulting in an even force application over a longer period of time. This is particularly advantageous compared to known solutions, which are based, for example, on multi-layered fabrics with undefined alignment of the individual fabric threads. It is also advantageous that mechanically very defined ribbon fabrics are used, which can be designed to be very flexible and adaptable in terms of their basic mechanical properties. In particular, the use of woven ribbon fabrics can reduce the thrombogenicity of the entire structure, as any shear forces on the blood to be transported are reduced. Blood compatibility can also be improved by coating the fabric with blood-compatible biomaterials or cells using spraying, electrospinning or dipping. The use of a precisely alignable tape fabric with defined thread alignment also achieves a continuous and uniform load distribution in both radial and tangential directions in the fabric. Without being bound by theory, it is also assumed that the special alignment of the weft threads in particular leads to a mechanically and fluid mechanically particularly favorable, smooth and firm cusp edge, which enables a more uniform closing process over a longer period of time. Overall, this arrangement achieves a longer fatigue strength under in-vivo conditions, a better flow profile and improved closing behavior.

The valve prosthesis according to the invention is suitable for implantation in the human or animal body. The embodiment according to the invention can replace the animal pericardial fabric frequently used in the field of human medicine in all embodiments of bio-valves. This applies to surgically insertable valves as well as minimally invasive implantable, so-called TAVI valves. The prosthesis is in contact with fluids of the human or animal, preferably mammalian, body, whereby the prosthesis has movable cusps or valves which can move reversibly from an open to a closed position as a function of the applied pressure. In the closed position, in particular the applied hydrostatic pressure is resisted and the passage of fluids, such as blood, is prevented. The valve prosthesis can, for example, be a heart valve, a venous valve or another prosthesis based on the same valve function principle with movable closure cusps.

The valve prosthesis comprises at least one valve stent consisting of an annular valve base. The valve stent forms the mechanical framework of the prosthesis and comprises an approximately round valve base. The valve base can be in the form of a ring, a short cylinder or a tube, with the embodiments differing in their ratio of diameter and longitudinal cylinder expansion. The diameter of the valve base can be between 5 mm and 4 cm, for example. For example, the annulus diameter for aortic valves can be 19, 21, 23, 25, 27 or 29 mm. The length of the prosthesis can be between 10 mm and 5 cm, for example, and the thickness of the tubes or cylinder wall can be between 0.1 mm and 2 mm.

At least two commissural struts are arranged on this valve base or these extend from the base. For example, it is possible for the struts and the base to be formed in one piece. Two or more struts can extend from the base. For example, the stent may have two, three or six struts which, in interaction with the fabric, define the number of movable cusps, since the ribbon fabric moves relative to the commissural struts during the biomechanical movement and thus contributes to the closure of the valve base surface. In the case of multiple commissural struts, these extend in the same direction from the valve base. Aortic or pulmonary valves can conveniently have three commissural struts. The valve stent can, for example, be made of carbon, a biocompatible plastic or metals or metal alloys such as Nitinol, Elgiloy, titanium or stainless steel. Suitable plastics can include polyethylene, polypropylene, polyurethanes, polysulfones, polyether sulfones, polyaryl sulfones and polyetherimides.

Furthermore, the valve prosthesis comprises valve cusps arranged on the valve stent, wherein the valve cusps are formed from one or more woven ribbon fabrics. A ribbon fabric is a textile fabric consisting of at least two separate thread systems, warp and weft. The warp and weft run at approximately 90° to each other. Warp and weft can consist of one or more threads. Preferably, only one thread can be used to form the fabric for warp and weft. The ribbon fabric can be produced as a whole by weaving. In particular, the ribbon fabric and the cusp edge can be produced by a continuous weaving process, in which case the weft reversal at the last warp thread to form the free cusp edge is preferably 180°. However, it is also possible to produce the ribbon fabric by weaving and to realize the weft reversal by crocheting. In this case, the weft yarn reversal at the free cusp edge is preferably also 180°. A shuttle weaving machine, for example, can be used to form a cusp edge with 180° weft reversal. In a tape produced on this type of machine, the weft threads reverse their orientation by 180 degrees at the last warp thread, forming two identically configured tape edges. In the case of a ribbon produced on a needle loom, one ribbon edge can also be formed by a 180-degree weft thread reversal on the last warp thread and the opposite other ribbon edge can be formed by crocheting the weft thread. When crocheting, the weft thread performs a 180-degree weft thread reversal by looping around the neighbouring weft thread loop (instead of looping around the last warp thread). Both selvedge shapes, the woven selvedge and the crocheted selvedge, are suitable as free cusp edges and show a weft reversal in the sense of the invention.

The ribbon fabric is attached to the stent either inside or outside the base of the valve to form the cusps. The entire cusp surface can be formed by one or more ribbons of fabric. If only one fabric ribbon is used, the fabric ribbon can be placed around the stent or on the inside of the stent and the two remaining ribbon edges can be fixed mechanically or chemically against each other, against the valve stent itself or against each other and against the stent. In addition to the ribbon edges, the surfaces of the ribbon fabric itself can generally also be mechanically or chemically fixed against the stent, particularly in the area of the commissural struts. However, it is also possible to use more than one ribbon fabric. In this case, the individual strips of ribbon fabric must be mechanically fixed against each other or against the stent. This design means that instead of a seamless tube, a bonding or clamping point always remains on the cusp structure. However, the cusp surfaces made from the ribbon fabric can be single-layered in large areas, i.e. no double layer consisting of more than one warp and weft thread is formed on the predominant cusp surface. However, the cusp surfaces are folded, partially superimposed, sewn, welded or clamped in the overlapping area of individual tape fabrics. Preferably, the entire cusp surface can be made up of one, two, three, four or up to 10 individual ribbon fabrics. In particular, it is not in accordance with the invention that the tape fabric is a seamless tubular fabric. The ribbon fabric can, for example, be woven in a rectangular shape. However, it is also possible for other shapes to be produced from the rectangular cut-to-length ribbon fabric, for example by cutting near the lower edge of the ribbon, and for these shapes to be fixed to the stent. For this purpose, it can be helpful to mechanically protect the cutting edges against fraying, for example by gluing. However, it is also possible to fix these shapes directly to any shape of the stent using ultrasonic welding.

Furthermore, weft and/or warp yarns can be composed of different multifilament yarns. Yarns with a core/sheath structure can also be used. In general, filaments can be selected from a group consisting of synthetic fibers such as polypropylene, nylon, polyesters, polyethylene, polyvinylidene fluoride, aramids and polyaramides. Some or all of the fibers may consist of biodegradable polymers, e.g. based on polyglycolic acid, polylactic acid or copolymers thereof.

In relation to the axis of symmetry of the annular valve base, the ribbon fabric comprises approximately tangential warp threads and approximately axial weft threads. Reference is made to the symmetry drawings of the boiler formula to define the course of the warp and weft threads. The weft yarns run axially on the cylinder surface when the valve is fully open, i.e. when the fabric is straight. If the valve is closed, the weft yarns run radially in the cusp. Such a definition also results when considering the symmetry axis of the stent. The axis of symmetry of the annular valve base can, for example, be the center point vector of the annular valve base. The commissural struts of the stent are usually aligned parallel to this center vector. The warp threads run tangentially around this axis of symmetry, i.e. approximately in a circle around the annular valve base. The warp threads thus also extend circularly around the valve stent in the area of the valve base between the commissural struts in cases where several commissural struts are present. The warp threads run approximately tangentially in cases where the angle between the individual warp thread and the plane running through the annular valve base is less than or equal to 20°, preferably less than or equal to 10°, more preferably less than or equal to 5°. Preferably, the warp threads form an angle of less than or equal to 5°, preferably 0°, to the plane through the annular valve base on average over the fabric. The orientation of the warp threads therefore essentially follows the symmetry of the fabric. The weft threads, on the other hand, run in an axial direction when the cusp is open. This means that the weft threads run parallel to the center vector of the valve base and therefore parallel to the commissure struts. The weft yarns therefore run approximately at right angles to the warp yarns. The weft threads run approximately axially in those cases in which, when the valve is open, the angle between the individual weft thread and the center vector of the ring base is less than or equal to 20°, preferably less than or equal to 10°, more preferably less than or equal to 5°. Preferably, the weft yarns form an angle of less than or equal to 5°, preferably 0° to the center point vector on average over the fabric. The orientation of the warp and weft threads used here is determined for the non-closed, i.e. open, state of the valve prosthesis.

In the fabric, at least the weft threads adjacent to the end of the fabric opposite the valve base reverse their orientation and form a free cusp edge. According to the invention, the weft threads run approximately axially and reverse their orientation, for example at the last warp thread, for example by folding them over, and run axially “back” again. By folding over all or only some of the weft threads, a compact edge is formed which provides a mechanically stable and flat finish to the fabric. In this respect, the individual cusps can lie “cusp edge to cusp edge” against each other in the closed state, which contributes to increased tightness in the closed state. Furthermore, the combination with the alignment of the weft yarns according to the invention results in an improved flow profile, which is particularly advantageous compared to other, angled yarn alignments and contributes, for example, to reduced flow resistance and a reduced shear gradient. The alignment and this arrangement also prevent or minimize undesirable turbulence. This can also improve the longevity of the arrangement. This cusp edge design is particularly advantageous in the area of the commissure strut edge, as it provides a favorable contour for the flow of the liquid medium. To form a free cusp edge, preferably more than 70%, further preferably more than 80%, further preferably more than 95% and further preferably 100% of the weft yarns at the edge can reverse their direction.

In a preferred embodiment of the valve prosthesis, the ribbon(s) can be arranged parallel to the axis of symmetry of the valve stent and the warp threads of the ribbon(s) can be aligned approximately radially in the upper area of the commissural struts. For the attachment of the ribbon fabric(s) to the valve stent or for the attachment of the edges of one ribbon fabric, it has proven to be particularly helpful for the attachment to be parallel to the axis of symmetry of the valve stent. The suture, clamping or adhesive point for fixing the ribbon mesh is therefore parallel to the center vector of the valve stent. Accordingly, it runs from “bottom” to “top”. This type of fixation enables a particularly homogeneous distribution of the mechanical loads occurring in the area of this fixation point. Depending on the number of webbings used to form the cusps, there may be one or more fixing points. In these cases, with several fixation points, this type of fixation is used for each individual point. It is also particularly advantageous for load absorption that the warp threads in the ribbon fabric are radially aligned, especially in the upper area of the commissural struts of the valve prosthesis. The upper area of the commissural struts results from the upper third of the extension of the commissural struts including the base of the valve. In this area, the warp threads are approximately radially aligned, which means that the angular deviation of the average alignment of the warp threads in this area relative to a theoretical plane through the base of the valve is less than or equal to 15°, preferably less than or equal to 10° and furthermore preferably less than or equal to 5°.

In a further preferred embodiment of the valve prosthesis, the valve stent can have three commissural struts and the valve cusps can be formed from three adjacent ribbons. A structure with three commissural struts and three individual ribbon fabrics, which together subsequently form the individual valve cusps, has proven to be particularly suitable for the formation of aortic or pulmonary valves. This structure can result in particularly mechanically resilient and durable prostheses which, if the suture points are arranged on the individual commissural struts, enable particularly suitable force distribution and transmission through the commissural struts to the valve base.

In a further preferred aspect of the valve prosthesis, the valve stent can have two commissural struts and the valve cusps can be formed from two adjacent ribbons. A structure with two commissural struts and two individual ribbon fabrics, which together subsequently form the individual valve cusps, has proven to be particularly suitable for the formation of venous valves. This structure can result in particularly mechanically resilient and durable prostheses which, if the sutures are arranged on the individual commissural struts, enable particularly suitable force distribution and transmission through the commissural struts to the base of the valve.

According to a preferred characteristic of the valve prosthesis, the valve cusps can have one or more additional layers of fabric at least partially in the area where the ribbon fabrics are arranged in relation to each other. In addition to fixing the individual ribbon fabrics to each other or to the valve stent itself, it may also be advantageous to attach further reinforcing layers at these points. These additional reinforcing layers can cover the belt fabric in these partial areas and be connected to the underlying belt fabric, for example by welding, sewing, weaving or gluing. These additional layers can further reinforce areas subject to particularly high mechanical stress and further define the force profile for transferring the occurring forces into the stent. In this respect, this measure also allows the force absorption profile of the stent to be flexibly adapted to the implantation situation at hand. In this embodiment, it is not in accordance with the invention that the entire ribbon fabric and in particular the free cusp edge are covered by the additional layers of fabric. Preferably, these fabric layers are only located in the area of the joint edges of the ribbon fabric or in the area of the commissure struts. These additional fabric layers preferably cover less than 20%, more preferably less than 15% and further preferably less than 10% of the entire cusp area.

In a further preferred embodiment of the valve prosthesis, the valve stent can have a mechanical clamping device in the area of the commissural struts for mechanical fixation of the fabric to each other and to the valve stent. The design of a mechanical clamping device for the ribbon fabric on the valve stent in conjunction with folding and suturing of the ribbon ends has proven to be particularly suitable for mechanically secure and therefore durable attachment of the ribbon fabric(s) to the valve stent. Valve prostheses can be realized which can provide a particularly high number of closing cycles under in vivo conditions without mechanical failure. In these cases, it can also be particularly advantageous that the clamping device fixes both the ribbon fabric on the prosthesis and the ribbon fabric(s) against each other. In this respect, a fixed mechanical arrangement of the ribbon fabric against the valve stent and against other existing ribbon fabrics to form the valve cusps is achieved by a fixation.

Within a preferred aspect of the valve prosthesis, the ribbon fabric(s) can be arranged only on the outside or inside of the valve stent, whereby the ribbon fabric does not contact the entire surface of the valve stent. By using ribbon fabric to form the valve cusps, it is possible in principle for the ribbon fabric to be placed around the outside of the valve base or the entire valve stent or to be fixed from the inside against the valve base and the entire valve stent. It has also been found that it is unnecessary for the inserted ribbon fabric(s) to contact the entire surface of the stent. For example, it is possible that the ribbon fabric is only attached to certain surface areas of the valve base or the commissural struts. In particular, these attachment points are not located at the lower end of the valve base, but can preferably extend at the upper end of the valve base in the direction of the commissural struts. In these embodiments, the lower part of the valve base is therefore not covered by a webbing. This configuration can be achieved by welding or gluing the ribbon fabric to the upper edge of the valve base. In this case, too, the lower part of the valve base is not covered by a woven ribbon fabric. This type of mechanical fixation can create further free space in the design of the lower valve base, which can lead to the valve stent as such or, in particular, the lower valve base being able to be precisely adapted to the installation situation without having to take into account a ribbon fabric in this area.

In a further preferred embodiment of the valve prosthesis, the valve base can have further anchoring structures all around, with the ribbon fabric(s) being arranged on the valve base at least in the area of the anchoring structures. It is also possible that fastening structures are attached to the lower valve base, with which the individual ribbon fabric(s) are mechanically connected. This can be done, for example, by fixing the ribbon fabric to the fastening structures. The mechanical fixation can create further free space in the design of the lower valve base, which can lead to the valve stent as such or, in particular, the lower valve base being able to be precisely adapted to the installation situation without having to take a ribbon fabric in this area into account.

In a further embodiment of the valve prosthesis, the ribbon fabric can have a weft yarn density of greater than or equal to 20 weft yarns/cm and less than or equal to 90 weft yarns/cm and a warp yarn density of greater than or equal to 60 warp yarns/cm and less than or equal to 180 warp yarns/cm. The use of ribbon fabric with the above-mentioned thread densities has proven to be particularly suitable for the formation of valve prostheses with a particularly high mechanical strength of the cusp surfaces. This makes it possible to obtain valve prostheses that can withstand very large load changes over long periods of time. This can have a particularly favorable influence on the in vivo suitability of the valve prosthesis. Higher densities may be unsuitable as the ribbon fabric can become too stiff in these cases. Lower thread densities can be disadvantageous, as in these cases the mechanical strength of the entire ribbon fabric could become too low. Particularly preferably, the weft yarn density can be greater than or equal to 25 weft yarns/cm and less than or equal to 80 weft yarns/cm, furthermore greater than or equal to 30 weft yarns/cm and less than or equal to 70 weft yarns/cm. Particularly preferably, the warp yarn density can be greater than or equal to 70 warp yarns/cm and less than or equal to 160 warp yarns/cm, furthermore preferably greater than or equal to 80 warp yarns/cm and less than or equal to 140 warp yarns/cm.

In a preferred embodiment of the valve prosthesis, the warp as well as the weft yarns of the tape fabric can consist of a multifilament yarn, wherein the multifilament consists of greater than or equal to 10 and less than or equal to 30 individual filaments and the individual filaments have a fineness of greater than or equal to 20 dtex and less than or equal to 90 dtex. In particular, the use of multifilaments of the above dimensions may be suitable for obtaining elastic and tension-resistant valve cusps which can withstand a high mechanical load without breaking, even at the mechanically exposed connection points. In this respect, the use of multifilament tape fabrics can contribute to improving the durability of the valve cusps. A larger number of filaments can be disadvantageous, as in these cases the yarn would either be too large or the diameter of the individual filaments would be too small. A smaller number of filaments could be disadvantageous, as in these cases the advantage of using a multifilament yarn cannot be fully utilized. The size of the individual filaments can be determined using DIN EN ISO 2060, for example.

In a further preferred embodiment of the valve prosthesis, the warp and weft yarns of the tape fabric can consist of different multifilament yarns, whereby the maximum tensile strength according to DIN EN ISO 2062 of the multifilament yarn of the warp yarns is greater than or equal to 10% and less than or equal to 60% higher than that of the weft yarns and the maximum tensile elongation according to DIN EN ISO 2062 of the multifilament yarn of the weft yarns is greater than or equal to 5% and less than or equal to 50% higher than that of the warp yarns. The structure of the valve cusps made of tape fabric provides durable valve cusps that can withstand high mechanical loads. The durability of the valve cusps made of woven tape can also be increased in particular by providing different mechanical properties for the different threads. It has proven to be particularly advantageous that the tensile strength of the warp yarns is higher than the tensile strength of the weft yarns. It is also advantageous for the elasticity of the weft threads to be greater than the elasticity of the warp threads. This combination in the asymmetry of the thread properties can once again significantly improve the mechanical resistance of the valve cusps.

In a further preferred embodiment of the valve prosthesis, the individual filaments of the weft threads can be twisted, whereby the twisting is greater than or equal to 10 turns/m and less than or equal to 600 turns/m. In order to improve the mechanical and, in particular, the elastic properties of the threads used, it has proved to be particularly advantageous for the weft threads in particular to be twisted into one another to form the woven tape fabric. The twisting allows a particularly reversible load introduction and load transfer into the fibers, which is reflected in an improved long-term behavior of the entire valve cusp under mechanical load.

Further according to the invention is the use of the valve prosthesis according to the invention as a textile scaffold for cell colonization. Surprisingly, it has also been shown that the structure according to the invention is highly suitable for serving as a matrix for the colonization of cells. Without being bound by theory, it is highly probable that two different factors contribute to this suitability. Firstly, it is assumed that the structure of the stent according to the invention with the special orientation of the threads in the fabric is conducive to colonization and, secondly, that the structure and orientation of the threads in the fabric is particularly suitable for biologically compatible yarns. The combination of structure and choice of material results in a substrate with a resulting porosity that is particularly suitable for cell colonization. The fabric can therefore serve as a textile scaffold in the sense of fabric engineering. Preferably, different cell types can be colonized in combination with a biofunctional coating, for example a liquid, polymerizable matrix (hydrogel). Other biologically active substances, such as scaffold proteins or fibrin, can be embedded in this coating. Colonization can then be carried out with the body's own cells such as myofibroblasts or endothelial cells. The coating or colonization with cells can take place before or after mounting the fabric on the valve stent. In the case of pre-coating/colonization, the tissue-engineered fabric can be attached to the valve stent using one or more clamping rings, whereby the common practice of manual suturing is advantageously dispensed with.

In a preferred embodiment of use, the valve stent can be used as a matrix for cell colonization in a two-step process. For example, the fabric can consist of medically suitable polyethylene terephthalate (PET) multifilament threads, whereby the fabric with the orientation of the threads according to the invention can have a Young's modulus in the range of 100 MPa. In a first step, the fabric can be coated with a fibrin gel which, after polymerization, provides a suitable basis for the mechanical and biological anchoring of different cell types on the fabric. This design also enables the biological functionalization of valve stents that are exposed to high hydrostatic pressures. In a second step, isolated cells or mixed populations of different cell types, such as fibroblasts and/or smooth muscle cells, can be applied to this fibrin base and cultivated in a bioreactor. A dense cell population can develop, which is mechanically held securely on the fabric by the gel. By colonizing the stent with the colonization matrix according to the invention, thromboembolic complications can be avoided or reduced, which would otherwise lead to damage to the blood components due to unfavourable shear stress and a non-physiological flow profile. Overall, the structure with colonization therefore leads to a long-term stable and hemocompatible valve stent.

In a further preferred embodiment of use, the material of at least one thread of the fabric may be selected from the group consisting of oriented poly(ester urethane)urea, oriented poly(ε-caprolactone), poly(ethylene glycol) (PEG), PEG diacrylate, (PEGDA), poly(L/DL)-lactide (PLDL) multifilaments, poly-lactide-co-glycolide (PLGA), or mixtures of at least two types from this group. The materials can also be produced by electrospinning. In combination with the structure according to the invention, these materials have proven to be particularly suitable for forming an extracellular matrix after cell colonization. In addition, the structure of the stent and the colonization as described can be used to provide constructs that differ significantly from the standard engineering materials in terms of their mechanical and biological properties. In particular, the mechanical resilience and hemo-compatibility can be significantly improved.

In a further preferred embodiment of use, PLDL multifilament yarns with a titer of 120 dtex made from 30 individual filaments can be used as yarns for the warp and weft threads. The yarns can also be coated with a thin layer of polylactide-co-glycolide consisting of 85% lactide and 15% glycolide. This can improve the adhesion of the fibrin base.

### Example

An artificial aortic valve is constructed using a stent consisting of a ring-shaped lower valve base and three commissural struts. Three individual ribbons serve as cusps, which are clamped against the stent and against each other in the area of the commissural struts. The design of the clamping is shown schematically in the figures. The ribbon fabric was woven from a yarn with the following specifications:
Warp and weft threads of the same multifilament yarn
Thread composition PET-polyethylene terephthalate
Fineness of the single filament 49 dtex
16 filaments per thread
16 single filaments twisted with 400 turns/meter
Tape fabric width 25 mm with 284 warp threads
46 weft yarns/cm

This structure was subjected to an in-vitro load test in accordance with DIN 5840. The pressure difference (opening/closing) is 100 mmHg and the load is changed at a frequency of 600/minute. Even after 10 million load cycles, the aortic valve shows no signs of mechanical fatigue of the cusps or the entire stent structure.

Further advantages and advantageous embodiments of the objects according to the invention are illustrated by the figures and explained in the following examples. It should be noted that the figures are descriptive only and are not intended to limit the invention in any way.

It shows the

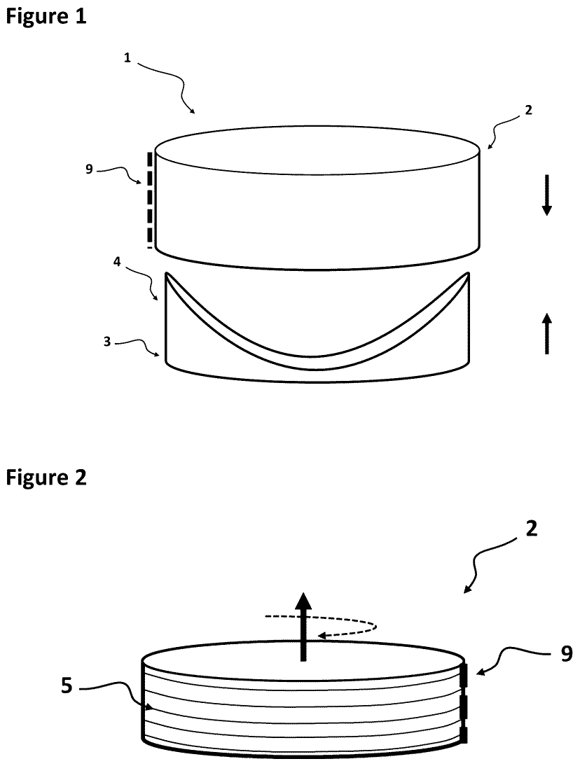

FIG. 1 schematic of an arrangement according to the invention consisting of band fabric and valve stent;

FIG. 2 schematic of the warp-thread alignment in the belt fabric according to the invention;

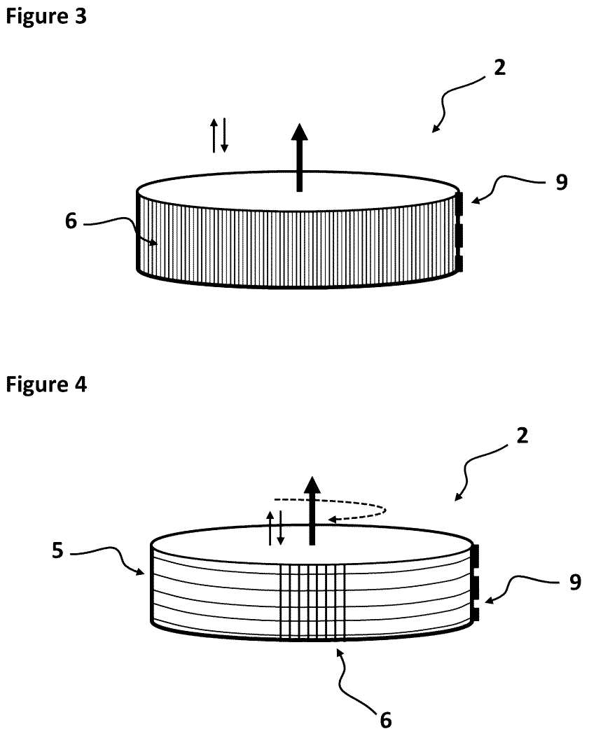

FIG. 3 schematic of the weft yarn alignment in the belt fabric according to the invention;

FIG. 4 schematic of the warp and weft yarn alignment in the belt fabric according to the invention;

FIG. 5 schematic of the formation of two “free” cusp edges by weft reversal;

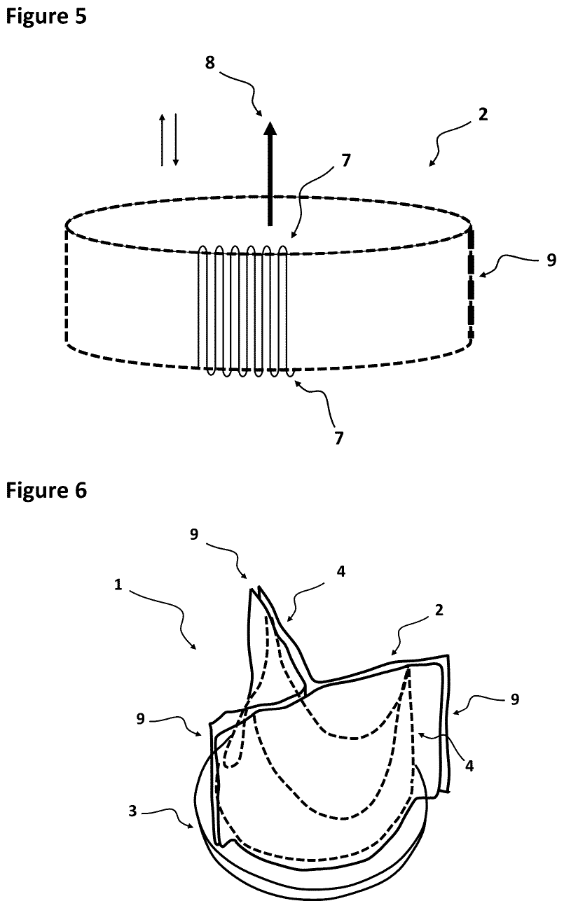

FIG. 6 schematic of an embodiment of an aortic valve according to the invention;

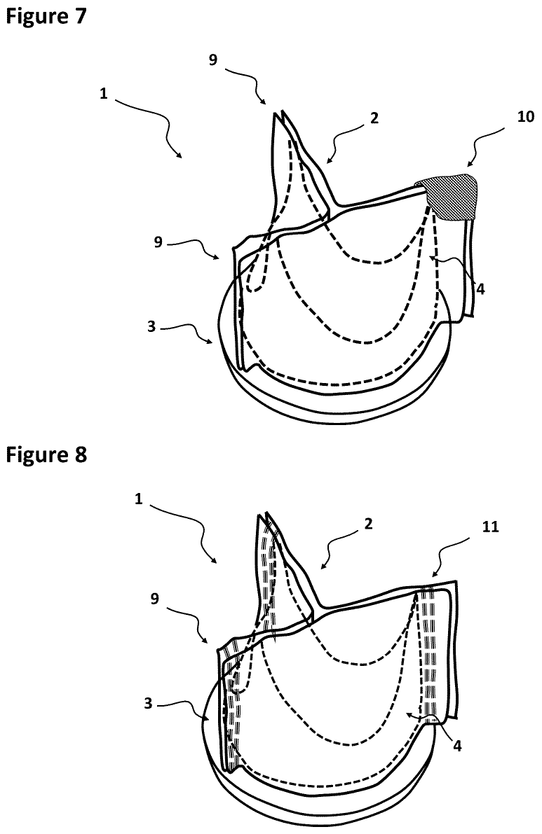

FIG. 7 schematic of an embodiment of an aortic valve with additional fabric stabilization according to the invention;

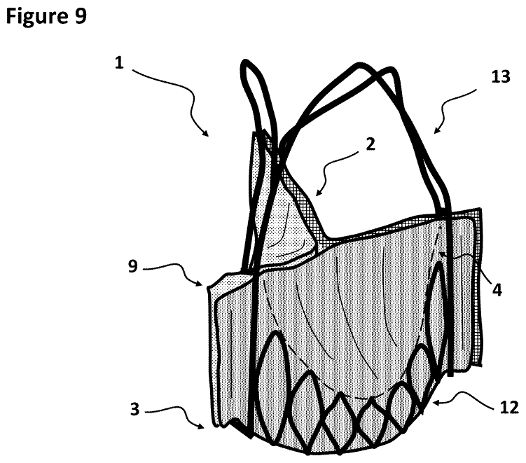

FIG. 8 schematic of an embodiment of an aortic valve according to the invention with additional stitching in the ribbon fabric;

FIG. 9 schematic of an embodiment of an aortic valve according to the invention with retaining structures on the valve ring.

FIG. 1 shows an arrangement 1 according to the invention consisting of ribbon fabric 2 and valve stent with valve base 3 and commissure strut 4. The valve base 3 of the stent carries two commissure struts 4 aligned in the same direction. In this figure, the arrangement consisting of ribbon fabric 2 and valve stent is achieved by placing the one-piece ribbon fabric 2 on the outside of the commissure struts 4 and the annular valve base 3. However, it is also possible in principle for the ribbon fabric 2 to be applied to the valve base 3 from the inside. The ribbon fabric 2 is placed around the annular valve base 3 and the edges of the ribbon are attached to each other or to the stent 1 or to both. The tape edges that run parallel to the axis of symmetry of the stent 1 are attached. The attachment is indicated in this figure by a clamping, suture or adhesive point 9, which fixes the ribbon fabric 2 to the stent. This point 9 can mean a fixation of the ribbon fabric to itself or a fixation of the ribbon fabric 2 at this point on the valve stent 1. For this purpose, the stent can, for example, have a clamping device for holding both edges of the ribbon fabric at this point. The valve cusps are therefore not seamless.

FIG. 2 shows the orientation of the warp threads 5 in the ribbon fabric 2 according to the invention. The warp threads 5 run tangentially to the axis of symmetry of the stent, which is illustrated by the center vector of the annular valve base (not shown).

FIG. 3 shows the orientation of the weft yarns 6 in the ribbon fabric 2 according to the invention. The weft yarns 6 run axially or parallel to the symmetry axis of the stent, which is illustrated by the center vector of the annular valve base 3 (not shown).

FIG. 4 shows the orientation of the warp yarns 5 and weft yarns 6 in the band fabric 2 according to the invention. The warp yarns 5 run tangentially, whereas the weft yarns 6 run axially or parallel to the axis of symmetry of the stent when the fabric is unfolded (i.e. the valve cusps are maximally open).

FIG. 5 shows the orientation of the weft yarns 6 in the ribbon fabric 2 according to the invention. In the open state, the weft yarns 6 run axially or parallel to the axis of symmetry 8 of the stent, whereby the weft yarns in this embodiment reverse their orientation at the upper and lower edge of the fabric 2 and form a “free” cusp edge 7 in each case. It is of course also possible that the edge in the area of the valve base 3 is not designed as a free cusp edge.

FIG. 6 schematically shows an embodiment according to the invention, for example in the form of an aortic valve. The stent 1 (shown dashed) has an annular valve base 3 and three commissural struts 4 on it. The valve cusps are formed from three individual band fabrics 2, which are clamped against each other and against the valve ring in the area of the commissural struts 4 9. In these areas, the band fabrics 2 can also be sewn, folded, welded or glued. Combinations of these joining techniques can also be used. At their upper edge, the woven strips 2 each form a free cusp edge 7.

FIG. 7 schematically shows an embodiment of an aortic valve according to the invention with additional fabric stabilization 10. The structure of the aortic valve is essentially described in FIG. 6. In addition, the cusps 2 made of ribbon fabric are stabilized with an additional fabric 10 in the area of the commissural struts 4. This fabric 10 can be used to increase the mechanical load-bearing capacity of the cusps 2 or to generate specific force dissipation profiles in the commissural struts 4.

FIG. 8 schematically shows an embodiment of an aortic valve according to the invention with additional stitching 11 or taping 11 in two adjacent ribbon fabric ends. The structure of the aortic valve is essentially described in FIG. 6. In addition to the basic structure, this figure shows that, in addition to a clamping 9 in the area of the commissural struts 4, the ribbon fabrics 2 can also be sewn 11 or glued 11 to each other or to each other. This can also improve the mechanical load-bearing capacity of the structure and the cusps in particular.

FIG. 9 schematically shows an embodiment of an aortic valve according to the invention with retaining structures 12 on the stent-shaped compressible and deployable valve ring 3. In this embodiment, the valve ring 3 consists of the commissural struts 4 and the retaining structures 12, to which the cusps of ribbon fabric 2, which in this embodiment lie on the inside, can be fixed. The individual woven tapes 2 can be fixed by folding, sewing, gluing, weaving or welding. This can ensure, for example, that the cusps do not extend to the lower end of the valve ring base 3.

1. A valve prosthesis for implantation in a human or animal body, comprising:
at least one valve stent (1) with an annular, lower valve base (3);
at least two commissural struts (4) extending from the annular, lower valve base (3); and
valve cusps (2) arranged on the valve stent,
characterized in that the valve cusps (2) consist of one or more woven ribbon fabrics with, in relation to an axis of symmetry (8) of the annular, lower valve base (3), approximately tangential warp threads (5) and approximately axial weft threads (6), wherein at least the axial weft threads (6) are located at an end of the ribbon fabric opposite the annular lower valve base to reverse their orientation and form a free cusp edge (7).

2. The valve prosthesis according to claim 1, wherein the one or more ribbon fabric are arranged parallel to the axis of symmetry (8) of the valve stent (1) and the warp threads (5) of the one or more ribbon fabrics are aligned approximately radially in an upper region of the commissural struts (4).

3. The valve prosthesis according to claim 1, wherein the valve stent (1) has three commissural struts (4) and the valve cusps (2) are formed from three ribbon fabrics arranged next to each other.

4. The valve prosthesis according to claim 1, wherein the valve stent (1) has two commissural struts (4) and the valve cusps (2) are formed from two ribbon fabrics arranged next to each other.

5. The valve prosthesis according to claim 1, wherein the valve cusps (2) at least partially comprise one or more further fabric layers (10) in a region of arrangement surfaces of the one or more ribbon fabrics relative to one another.

6. The valve prosthesis according to claim 1, wherein the valve stent (1) comprises a mechanical clamping device (9) in the region of the commissural struts (4) for mechanically fixing the one or more ribbon fabrics to each other and to the valve stent (1).

7. The valve prosthesis according to claim 1, wherein the one or more ribbon fabrics are arranged only on an outer or an inner side of the valve stent (1), wherein the one or more ribbon fabrics does not contact an the entire surface of the valve stent (1).

8. The valve prosthesis according to claim 1, wherein the annular lower valve base (3) comprises further anchoring structures (12) all around, wherein the one or more ribbon fabrics are arranged on the annular, lower valve base (3) at least in a region of the anchoring structures (12).

9. The valve prosthesis according to claim 1, wherein the one or more ribbon fabrics have a weft yarn density of greater than or equal to 20 weft yarns/cm and less than or equal to 90 weft yarns/cm and a warp yarn density of greater than or equal to 60 warp yarns/cm and less than or equal to 180 warp yarns/cm.

10. The valve prosthesis according to claim 1, wherein the warp as well as the weft yarns of the one or more ribbon fabrics consist of a multifilament yarn, wherein the multifilament yarn consists of greater than or equal to 10 and less than or equal to 30 individual filaments and the individual filaments have a fineness of greater than or equal to 20 dtex and less than or equal to 90 dtex.

11. The valve prosthesis according to claim 1, wherein the warp and weft yarns of the one or more ribbon fabrics consist of different multifilament yarns, wherein a maximum ten-sile force according to DIN EN ISO 2062 of the multifilament yarn of the warp yarns is greater than or equal to 10% and less than or equal to 60% higher than that of the weft yarns and a maximum tensile force elongation according to DIN EN ISO 2062 of the multifilament yarn of the weft yarns is greater than or equal to 5% and less than or equal to 50% higher than that of the warp yarns.

12. The valve prosthesis according to claim 1, wherein the individual filaments of the weft yarns are twisted, wherein the twisting is greater than or equal to 10 turns/m and less than or equal to 600 turns/m.
